(19) United States
(12) Patent Application Publication
(10) Pub. No.: US 2006/0014838 A1
(43) Pub. Date: Jan. 19, 2006

(54) Process for converting trans-sertraline to cis-sertraline

(76) Inventors: Sudhakar Valluri, West Godavari (IN); Sandeep Mohonty, Bhubaneshwar (IN); Srinivasa Rao Talasila, Krishna (IN); Chandra Mohan Reddy Devireddy, Kadapa (IN); Sambasiva Rao Lella, Krishna (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/180,325

(22) Filed: Jul. 13, 2005

Related U.S. Application Data
(60) Provisional application No. 60/587,802, filed on Jul. 14, 2004.

(51) Int. Cl. A61K 31/137; C07C 209/68

(52) U.S. Cl. 514/649; 564/336

(57) ABSTRACT

A process for preparing sertraline hydrochloride includes reacting trans-N-methyl-4-(3,4-dichlorophenyl)-1,2,3,4-tetrahydro-1-napthaleneamine with a base in an aqueous basic organic solvent.

### CROSS-REFERENCE TO RELATED APPLICATION

This application claims priority from copending U.S. Provisional Application No. 60/587,802 filed on Jul. 14, 2004, the entire content of which is incorporated herein by this reference.

### INTRODUCTION TO THE INVENTION

The present invention relates to a process for a conversion of trans-isomeric 4-(3,4-dichlorophenyl)-1,2,3,4-tetrahydro-N-methyl-1-napthaleneamine to cis-isomeric 4-(3,4-dichlorophenyl)-1,2,3,4-tetrahydro-N-methyl-1-napthaleneamine and is useful for preparation of the drug compound having the adopted name “sertraline hydrochloride,” which is chemically known as (1S-cis)-4-(3,4-dichlorophenyl)-1,2,3,4-tetrahydro-N-methyl-1-napthalenamine hydrochloride and has the Formula I.

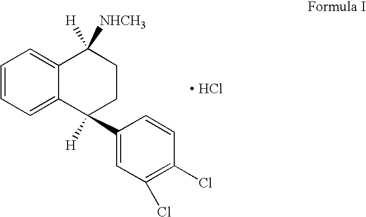

Sertraline hydrochloride is a selective serotonin reuptake inhibitor (SSRI) and is commercially available in pharmaceutical products sold using the trademark ZOLOFT™.

U.S. Pat. No. 4,536,518 describes the process for the preparation of sertraline. The process involves preparation of cis-(1S)(4S)-N-methyl-4-(3,4-dichlorophenyl)-1,2,3,4-tetrahydro-1-naphthaleneamine as a racemic mixture, starting from 3,4-dichlorobenzophenone. The isomeric mixture is then separated into its component parts by conventional means, e.g., by fractional crystallization of the hydrochloride salts or by column chromatography on silica gel of the corresponding free base. Resolution of the separated cis-racemate free base compound gives the desired cis-(1S)(4S)-enantiomer (sertraline).

U.S. Pat. No. 6,552,227 describes a process for the preparation of (+)-cis-sertraline having a cis/trans ratio greater than about 3:1. The process of said patent comprises hydrogenating sertraline-1-imine using a catalyst selected from the group consisting of palladium and platinum at a temperature of at least about 40° C.

U.S. Pat. No. 5,082,970 describes a process for converting trans-isomeric N-methyl-4-(3,4-dichlorophenyl)-1,2,3,4-tetrahydro-1-naphthaleneamine to cis-isomeric N-methyl-4-(3,4-dichlorophenyl)-1,2,3,4-tetrahydro-1-naphthaleneamine which involves treating trans-isomeric N-methyl-4-(3,4-dichlorophenyl)-1,2,3,4-tetrahydro-1-naphthaleneamine with a basic equilibration agent like potassium tertiarybutoxide in a reaction-inert polar organic solvent. The compound obtained by said process is mixture of cis- and trans-isomers in about a 2:1 ratio. Since the process involves use of inert polar organic solvents like tetrahydrofuran in the presence of alkali metal lower alkoxides, it is difficult to handle in a larger scale and difficult to recover solvents, and thus increases the cost of the production.

Hence, there is a need to develop a simple and cost effective process, which can be commercially viable. Therefore the present invention provides a cost-effective and commercially viable process, which involves conversion of the undesired trans-isomeric N-methyl-4-(3,4-dichlorophenyl)-1,2,3,4-tetrahydro-1-napthaleneamine to cis-isomeric N-methyl-4-(3,4-dichlorophenyl)-1,2,3,4-tetrahydro-1-napthaleneamine and is useful for preparation of sertraline hydrochloride.

### SUMMARY OF THE INVENTION

An aspect of present invention, therefore, is a process for making cis/trans-isomeric 4-(3,4-dichlorophenyl)-1,2,3,4-tetrahydro-N-methyl-1-napthalenamine having a cis/trans ratio of about 1:1, comprising reacting trans (1S,4R)-isomeric 4-(3,4-dichlorophenyl)-1,2,3,4-tetrahydro-N-methyl-1-napthalenamine and trans (1R,4S) isomeric 4-(3,4-dichlorophenyl)-1,2,3,4-tetrahydro-N-methyl-1-naphtha-lenamine in the presence or absence of corresponding cis-isomeric 4-(3,4-dichlorophenyl)-1,2,3,4- tetrahydro-N-methyl-1-napthalenamine of Formula II 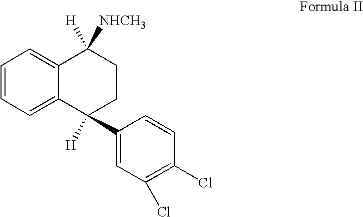
with a base in an aqueous basic solvent to give a cis/trans mixture wherein the cis- and trans-isomeric 4-(3,4-dichlorophenyl)-1,2,3,4-tetrahydro-N-methyl-1-naphalenalenamine are present in about a 1:1 weight ratio. The resultant cis/trans mixture of sertraline is further purified into pure (1S-cis)-4-(3,4-Dichlorophenyl)-1,2,3,4-tetrahydro-N-methyl-1-napthalenamine and is used for conversion into the hydrochloride salt.

The invention includes a process for preparing cis/trans-isomeric 4-(3,4-dichlorophenyl)-1,2,3,4-tetrahydro-N-methyl-1-napthalenamine comprising reacting trans-N-methyl-4-(3,4-dichlorophenyl)-1,2,3,4-tetrahydro-1-napthaleneamine with a base in an aqueous basic organic solvent.

The invention also includes a process for preparing cis/trans-isomeric 4-(3,4-dichlorophenyl)-1,2,3,4-tetrahydro-N-methyl-1-napthalenamine having a weight ratio of cis:trans isomers about 1:1, comprising reacting trans-N-methyl-4-(3,4-dichlorophenyl)-1,2,3,4-tetrahydro-1-napthaleneamine with an inorganic base in an aqueous basic organic solvent.

The invention further includes a process for preparing sertraline hydrochloride, comprising:

a) reacting trans-N-methyl-4-(3,4-dichlorophenyl)-1,2,3,4-tetrahydro-1-napthaleneamine with a base in an aqueous basic organic solvent, to form a racemic mixture of cis- and trans-amines;

b) reacting the mixture of cis- and trans-amines with hydrochloric acid; and

c) recovering solid sertraline hydrochloride.

### DETAILED DESCRIPTION OF THE INVENTION

In one aspect of the present invention, therefore, there is provided a process for making a cis/trans-isomeric 4-(3,4-dichlorophenyl)-1,2,3,4-tetrahydro-N-methyl-1-napthalenamine having a cis:trans ratio of about 1:1 comprising reacting trans (1S,4R)-4-(3,4-dichlorophenyl)-1,2,3,4-tetrahydro-N-methyl-1-napthalenamine and trans (1R,4S)-4-(3,4-dichlorophenyl)-1,2,3,4-tetrahydro-N-methyl-1-napthalenamine in the presence or absence of the corresponding cis- isomeric 4-(3,4-dichlorophenyl)-1,2,3,4-tetrahydro-N-methyl-1-napthalenamine of Formula II 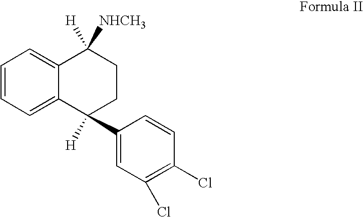
with a base in an aqueous basic solvent to give a cis/trans mixture wherein the isomers are present in a ratio of 1:1 by weight. The resultant cis/trans mixture is further purified to yield pure (1S-cis)-4-(3,4-Dichlorophenyl)-1,2,3,4-tetrahydro-N-methyl-1-napthalenamine and is used for conversion into the hydrochloride salt of Formula I.

Reaction of trans (1S,4R)-4-(3,4-dichlorophenyl)-1,2,3,4-tetrahydro-N-methyl-1-napthalenamine and trans (1R,4S)-4-(3,4-dichlorophenyl)-1,2,3,4-tetrahydro-N-methyl-1-napthalenamine in the presence or absence of the corresponding cis-isomer of sertraline of Formula II with a base is carried but in an aqueous basic solvent at a suitable temperature to afford a cis/trans mixture wherein the amount of cis-isomer of Sertraline is 1:1 on a weight basis. The resultant mixture of compounds is further purified into pure (1S-cis)-4-(3,4-Dichlorophenyl)-1,2,3,4-tetrahydro-N-methyl-1-napthalenamine and finally converted to its hydrochloride salt of Formula I.

Suitable aqueous basic solvents include but are not limited to mixtures of water and basic solvents such as N-methylpyrrolidinone, pyridine, lutidine, N,N-dimethylformamide, N,N-dimethylacetamide, triethylamine and the like; or mixtures thereof in various proportions. Pyridine is a particularly useful basic solvent. Lutidine exists in different configurations, including the compounds 2,3-dimethylpyridine, 2,4-dimethylpyridine, 2,5-dimethylpyridine, 2,6-dimethylpyridine, 3,4-dimethylpyridine, and 3,5-dimethylpyridine; any of these can be used in the invention.

Suitable bases for the process include but are not limited to organic bases such as methylamine, dimethylamine, triethylamine, di-isopropylethylamine, and the like, or inorganic bases such as sodium hydroxide, potassium hydroxide, sodium carbonate, sodium bicarbonate, potassium carbonate, potassium bicarbonate, and the like; sodium hydroxide or potassium hydroxide or their mixtures have been found to be particularly useful.

The process is normally carried in an aqueous basic solvent at a temperature in the range of about 20° C. to about 150° C. or at a reflux condition, and preferably in the range of from about 110° C. to about 130° C. until the desired conversion to the constant 1:1 (by weight) cis/trans-mixture is substantially obtained. This reaction is can require as long as about 30 hours, or 24 to 25 hours.

After completion of the reaction, pure racemic cis-N-methyl-4-(3,4-dichlorophenyl)-1,2,3,4-tetrahydro-1-naphthaleneamine or the pure chiral cis-(1S)(4S)-enantiomer thereof, is readily produced from the reaction mixture by conventional means, such as by first removing the solvent via evaporation under reduced pressure and then dissolving the residue in an aromatic hydrocarbon solvent such as toluene, xylene and the like, followed by washing and removal of the solvent under reduced pressure to afford the desired 1:1 (by weight) cis/trans product as a residual oil. The residual oil is thereafter dissolved in an alcoholic solvent such as methanol, ethanol, isopropanol, n-butanol, tertiary butyl alcohol and the like, and treated with an aqueous hydrohalide, like hydrogen chloride, and the desired cis-amine precipitates from solution as its crystalline hydrohalide salt while the corresponding trans-amine salt remains in solution. In this manner, a starting material such as unwanted racemic trans-N-methyl-4-(3,4-dichlorophenyl)-1,2,3,4-tetrahydro-1-naphthaleneamine or mixtures of it with the cis-isomer are expeditiously converted to pure crystalline racemic cis-N-methyl-4-(3,4-dichlorophenyl)-1,2,3,4-tetrahydro-1-naphthaleneamine hydrochloride through the aforesaid 1:1 (by weight) cis/trans mixture.

Therefore, the process of the present invention provides a method to convert undesired trans-isomeric N-methyl-4-(3,4-dichlorophenyl)-1,2,3,4-tetrahydro-1-naphthaleneamine to the desired pure cis-isomer and thus to effectively recycle the undesired trans-isomer into the desired cis-isomer. The achievement, in turn, represents an improvement over prior art methods in view of the decreased time cycle and ease of operation, and the decrease in costs of manufacture.

The following examples are only illustrative of certain aspects of the invention and are not intended to limit the scope of the invention.

### EXAMPLE 1

Preparation of Pure Racemic Cis-N-Methyl-4-(3,4-Dichlorophenyl)-1,2,3,4-Tetrahydro-1-Napthalenamine Hydrochloride of Formula (I)

200 g (0.654 moles) of mixture of racemic trans-isomeric 4-(3,4-dichlorophenyl)-1,2,3,4-tetrahydro-N-methyl-1-napthalenamine, 1000 ml of pyridine, 180 g (4.92 moles) of potassium hydroxide and 76 ml of water were charged to a clean and dry round bottom flask. The resultant reaction mixture was heated to about 115-120° C. for about 20-25 hours. Solvent from the reaction suspension was distilled at about 80° C. under reduced pressure and the resulting residue was dissolved in 1000 ml of toluene. Resultant reaction solution was washed with (5×668 ml) water and the solvent was distilled off at about 80° C. under reduced pressure, resulting in a residual oil containing a 1:1 mixture of racemic cis- and trans-isomeric 4-(3,4-dichlorophenyl)-1,2,3,4-tetrahydro-N-methyl-1-napthalenamine. The obtained residual oil was dissolved in methanol (1000 ml) and subsequently the resultant solution was treated with aqueous hydrochloric acid (100 ml, 32-34% w/w) to afford the hydrochloride salt of the cis-isomeric 4-(3,4-dichlorophenyl)-1,2,3,4-tetrahydro-N-methyl-1-napthalenamine. The separated solid was filtered to afford 103 g of title compound. The trans-amine remained in the filtrate.

### EXAMPLE 2

Alternative Preparation of Pure Racemic Cis-N-Methyl-4-(3,4-Dichlorophenyl)-1,2,3,4-Tetrahydro-1-Napthalenamine Hydrochloride of Formula (I)

A mixture of racemic trans-isomeric 4-(3,4-dichlorophenyl)-1,2,3,4-tetrahydro-N-methyl-1-napthalenamine 20.0 g (0.0654 moles), 100 ml of pyridine, 13 g (0.325 moles) of sodium hydroxide and 7.6 ml of water were charged to a clean and dry round bottom flask. The contents were heated to 115-120° C. for about 24 hours. The solvent from the reaction mass was distilled under reduced pressure and the resulting residue was dissolved in 100 ml of toluene. The reaction solution was washed with water (5×66.8 ml). Organic solvent was distilled under reduced pressure, which resulted in a residual oil containing a 1:1 mixture of racemic cis- and trans-isomeric 4-(3,4-dichlorophenyl)-1,2,3,4-tetrahydro-N-methyl-1-napthalenamine. The residual oil was dissolved in methanol (100 ml) and subsequently the resultant solution was treated with aqueous hydrochloric acid (10 ml, 32-34% w/w) to precipitate cis-isomeric 4-(3,4-dichlorophenyl)-1,2,3,4-tetrahydro-N-methyl-1-napthalenamine as its hydrochloride salt. The separated solid was filtered to get 10.3 g of pure racemic cis-N-methyl-4-(3,4-dichlorophenyl)-1,2,3,4-tetrahydro-1-napthalene hydrochloride. The trans-isomeric4-(3,4-dichlorophenyl)-1,2,3,4-tetrahydro-N-methyl-1-napthalenamine remained in the filtrate.

### EXAMPLE 3

Alternative Preparation of Pure Racemic Cis-N-Methyl-4-(3,4-Dichlorophenyl)-1,2,3,4-Tetrahydro-1-Napthalenamine Hydrochloride of Formula (I)

A mixture of racemic trans-isomeric 4-(3,4-dichlorophenyl)-1,2,3,4-tetrahydro-N-methyl-1-napthalenamine 20 g (0.0654 moles), 100 ml of pyridine, 6.5 g (0.162 moles) of potassium hydroxide, 9 g (0.161 moles) of sodium hydroxide, and 7.6 ml of water were charged to a clean and dry round bottom flask. The contents were heated to about 115-120° C. for about 20-25 hours. The solvent from the reaction mass was distilled completely at about 80° C. under reduced pressure and the resulting residue was dissolved in 100 ml of toluene. The resultant reaction solution was washed with water (5×66.8 ml). The organic solvent was distilled under reduced pressure, which resulted in a residual oil containing a 1:1 mixture of racemic cis- and trans-isomeric 4-(3,4-dichlorophenyl)-1,2,3,4-tetrahydro-N-methyl-1-napthalenamine. The residual oil was dissolved in methanol (100 ml) and was subsequently treated with aqueous hydrochloric acid (10 ml, 32-34% w/w) to precipitate cis-isomeric 4-(3,4-dichlorophenyl)-1,2,3,4-tetrahydro-N-methyl-1-napthalenamine as its hydrochloride salt. Separated solid was filtered to afford 9 g of pure racemic cis-N-methyl-4-(3,4-dichlorophenyl)-1,2,3,4-tetrahydro-1-napthalene hydrochloride. The trans-isomeric 4-(3,4-dichlorophenyl)-1,2,3,4-tetrahydro-N-methyl-1-napthalenamine remained in the filtrate.

### EXAMPLE 4

Alternative Preparation of Pure Racemic Cis-N-Methyl-4-(3,4-Dichlorophenyl)-1,2,3,4-Tetrahydro-1-Napthalenamine Hydrochloride of Formula (I)

A mixture of racemic trans-isomeric 4-(3,4-dichlorophenyl)-1,2,3,4-tetrahydro-N-methyl-1-napthalenamine 40 g (0.131 moles), 200 ml of pyridine, 36 g (0.643 moles) of potassium hydroxide and 15.2 ml of water were charged to a clean and dry round bottom flask. The contents were heated to about 115-120° C. for about 20-25 hours. The solvent from the reaction mass was distilled at about 80° C. under reduced pressure and the resulting residue was dissolved 200 ml of toluene. The solution was washed with water (5×134 ml). Organic layer was distilled off under reduced pressure, which resulted in a residual oil containing a 1:1 mixture of racemic cis- and trans-isomeric 4-(3,4-dichlorophenyl)-1,2,3,4-tetrahydro-N-methyl-1-napthalenamine. The residual oil was dissolved in isopropyl alcohol (200 ml) and subsequently treated with aqueous hydrochloric acid (20 ml, 32-34% w/w) to precipitate cis-isomeric 4-(3,4-dichlorophenyl)-1,2,3,4-tetrahydro-N-methyl-1-napthalenamine as its hydrochloride salt. Separated solid was filtered to afford 11 g of pure racemic cis-N-methyl-4-(3,4-dichlorophenyl)-1,2,3,4-tetrahydro-1-napthalene hydrochloride. The trans-isomeric 4-(3,4-dichlorophenyl)-1,2,3,4-tetrahydro-N-methyl-1-napthalenamine remained in the filtrate.

### EXAMPLE 5

Alternative Preparation of Pure Racemic Cis-N-Methyl-4-(3,4-Dichlorophenyl)-1,2,3,4-Tetrahydro-1-Napthalenamine Hydrochloride of Formula (I)

A mixture of racemic trans- isomeric 4-(3,4-dichlorophenyl)-1,2,3,4-tetrahydro-N-methyl-1-napthalenamine 40 g (0.131 moles), 200 ml of pyridine, 36 g (0.643 moles) of potassium hydroxide and 15.2 ml of water were charged to a clean and dry round bottom flask. The contents were heated to about 115-120° C. for about 20-25 hours. The solvent was distilled from the reaction mass at about 80° C. under reduced pressure and the resulting residue was dissolved in 200 ml of toluene. The solution was washed with water (5×134 ml). The organic layer was distilled under reduced pressure, which resulted in a residual oil containing a 1:1 mixture of racemic cis- and trans-isomeric 4-(3,4-dichlorophenyl)-1,2,3,4-tetrahydro-N-methyl-1-napthalenamine. The residual oil was dissolved in a mixture (200 ml) of isopropyl alcohol and tertiary butyl alcohol (80:20 by volume) and subsequently treated with aqueous hydrochloric acid (20 ml, 32-34% w/w) to precipitate cis-amine as its hydrochloride salt. Separated solid was filtered to afford 13 g of pure racemic cis-N-methyl 4-(3,4-dichlorophenyl)-1,2,3,4-tetrahydro-1-naphthalene hydrochloride. The trans-isomeric 4-(3,4-dichlorophenyl)-1,2,3,4-tetrahydro-N-methyl-1-napthalenamine remained in the filtrate.

### EXAMPLE 6

Alternative Preparation of Pure Racemic Cis-N-Methyl-4-(3,4-Dichlorophenyl)-1,2,3,4-Tetrahydro-1-Napthalenamine Hydrochloride of Formula (I)

A mixture of racemic trans-isomeric 4-(3,4-dichlorophenyl)-1,2,3,4-tetrahydro-N-methyl-1-napthalenamine 40 g (0.131moles), 200 ml of lutidine, 36 g (0.643 moles) of potassium hydroxide and 15.2 ml of water was placed into a reaction vessel. The contents were heated to 115-120° C. for about 20-25 hours. The solvent from the reaction mass was distilled at about 80° C. under reduced pressure and the resulting residue was dissolved in 200 ml of toluene. The solution was washed with water (5×134 ml). The solvent was distilled under reduced pressure, which resulted in a residual oil containing a 1:1 mixture of racemic cis- and trans-isomeric 4-(3,4-dichlorophenyl)-1,2,3,4-tetrahydro-N-methyl-1-napthalenamine. The residual oil was dissolved in isopropyl alcohol (200 ml) and was subsequently treated with aqueous hydrochloric acid (20 ml, 32-34% w/w) to precipitate cis-isomeric 4-(3,4-dichlorophenyl)-1,2,3,4-tetrahydro-N-methyl-1-napthalenamine as its hydrochloride salt. Separated solid was filtered to get 11.0 g of pure racemic cis-N-methyl-4-(3,4-dichlorophenyl)-1,2,3,4-tetrahydro-1-napthalene hydrochloride. The trans-isomeric 4-(3,4-dichlorophenyl)- 1,2,3,4-tetrahydro-N-methyl-1-napthalenamine remained in the filtrate.

We claim:

1. A process for preparing cis/trans-isomeric 4-(3,4-dichlorophenyl)-1,2,3,4-tetrahydro-N-methyl-1-napthalenamine comprising reacting trans-N-methyl-4-(3,4-dichlorophenyl)-1,2,3,4-tetrahydro-1-napthaleneamine with a base in an aqueous basic organic solvent.

2. The process of claim 1, wherein prepared cis/trans-isomeric 4-(3,4-dichlorophenyl)-1,2,3,4-tetrahydro-N-methyl-1-napthalenamine has a cis:trans ratio of about 1:1 on a weight basis.

3. The process of claim 1, wherein a base comprises an inorganic base.

4. The process of claim 1 wherein an inorganic base comprises sodium hydroxide, potassium hydroxide, sodium carbonate, sodium bicarbonate, potassium carbonate, potassium bicarbonate, or a mixture of any two or more thereof.

5. The process of claim 1, wherein a base comprises an organic base.

6. The process of claim 1 wherein a base comprises an organic base comprising methylamine, dimethylamine, triethylamine, di-isopropylethylamine, or a mixture of any two or more thereof.

7. The process of claim 1, wherein an aqueous basic organic solvent comprises N-methylpyrrolidinone, pyridine, lutidine, N,N-dimethylformamide, N,N-dimethylacetamide, triethylamine, or a mixture of any two or more thereof.

8. The process of claim 1, wherein an aqueous basic organic solvent comprises pyridine or lutidine.

9. The process of claim 9, wherein an aqueous basic solvent comprises pyridine.

10. A process for preparing cis/trans-isomeric 4-(3,4-dichlorophenyl)-1,2,3,4-tetrahydro-N-methyl-1-napthalenamine having a weight ratio of cis:trans isomers about 1:1, comprising reacting trans-N-methyl-4-(3,4-dichlorophenyl)-1,2,3,4-tetrahydro-1-napthaleneamine with an inorganic base in an aqueous basic organic solvent.

11. The process of claim 10 wherein an inorganic base comprises sodium hydroxide, potassium hydroxide, sodium carbonate, sodium bicarbonate, potassium carbonate, potassium bicarbonate, or a mixture of any two or more thereof.

12. The process of claim 10, wherein an aqueous basic organic solvent comprises N-methylpyrrolidinone, pyridine, lutidine, N,N-dimethylformamide, N,N-dimethylacetamide, triethylamine, or a mixture of any two or more thereof.

13. The process of claim 1, wherein an aqueous basic organic solvent comprises pyridine or lutidine.

14. A process for preparing sertraline hydrochloride, comprising:
a) reacting trans-N-methyl-4-(3,4-dichlorophenyl)-1,2,3,4-tetrahydro-1-napthaleneamine with a base in an aqueous basic organic solvent, to form a racemic mixture of cis- and trans-amines;
b) reacting the mixture of cis- and trans-amines with hydrochloric acid; and
c) recovering solid sertraline hydrochloride.

15. The process of claim 14, wherein a base is an inorganic base.

16. The process of claim 15 wherein an inorganic base comprises sodium hydroxide, potassium hydroxide, sodium carbonate, sodium bicarbonate, potassium carbonate, potassium bicarbonate, or a mixture of any two or more thereof.

17. The process of claim 15, wherein an inorganic base comprises sodium hydroxide or potassium hydroxide.

18. The process of claim 14, wherein an aqueous basic organic solvent comprises N-methylpyrrolidinone, pyridine, lutidine, N,N-dimethylformamide, N,N-dimethylacetamide, triethylamine, or a mixture of any two or more thereof.

19. The process of claim 14, wherein an aqueous basic organic solvent comprises pyridine or lutidine.

20. The process of claim 14, wherein the mixture of cis- and trans-amines has a weight ratio of cis:trans about 1:1.
